(19) United States
(12) Patent Application Publication
(10) Pub. No.: US 2004/0171160 A1
(43) Pub. Date: Sep. 2, 2004

(54) Method for producing a marker-free mutated target organism and plasmid vectors suitable for the same

(76) Inventors: Markus Pompejus, Freinsheim (DE); Corinna Klopprogge, Ludwigshafen (DE); Oskar Zelder, Speyer (DE); Wolfgang Liebl, Bovenden (DE)

Correspondence Address: LAHIVE & COCKFIELD, LLP., 28 STATE STREET, BOSTON, MA, 02109, US

(21) Appl. No.: 10/486,125

(22) PCT Filed: Jul. 24, 2002
(86) PCT No.: PCT/EP02/08231

(30) Foreign Application Priority Data
Aug. 6, 2001 (DE) 101 37 815.7

(51) Int. Cl. C12N 9/12; C12N 15/74; C12N 1/21

(52) U.S. Cl. 435/488; 435/252.33; 435/194

(57) ABSTRACT

The invention relates to a plasmid vector which does not replicate in a target organism, comprising the following components:

a) an origin of replication for a host organism which is different from the target organism,

b) at least one genetic marker,

c) where appropriate, a sequence section which makes possible the transfer of DNA via conjugation (mob sequence),

d) a sequence section which is homologous to sequences of the target organism and makes possible homologous recombination in the target organism,

e) a gene for a galactokinase under the control of a promotor.

The invention relates to a novel method for modifying the genome of Gram-positive bacteria, to these bacteria and to novel vectors. The invention particularly relates to a method for modifying corynebacteria or brevibacteria with the aid of a novel marker gene which has a conditionally negatively dominant action in the bacteria.

Corynebacterium glutamicum is a Gram-positive, aerobic bacterium which (like other corynebacteria, i.e. Corynebacterium and Brevibacterium species too) is used industrially for producing a number of fine chemicals, and also for breaking down hydrocarbons and oxidizing terpenoids (for a review, see, for example, Liebl (1992) “The Genus Corynebacterium”, in: The Procaryotes, Volume II, Balows, A. et al., eds. Springer).

Because of the availability of cloning vectors for use in corynebacteria and techniques for genetic manipulation of C. glutamicum and related Corynebacterium and Brevibacterium species (see, for example, Yoshihama et al., J. Bacteriol. 162 (1985) 591-597; Katsumata et al., J. Bacteriol. 159 (1984) 306-311; and Santamaria et al. J. Gen. Microbiol. 130 (1984) 2237-2246), genetic modification of these organisms is possible (for example by overexpression of genes) in order, for example, to make them better and more efficient as producers of one or more fine chemicals.

The use of plasmids able to replicate in corynebacteria is in this connection a well-established technique which is known to the skilled worker, is widely used and has been documented many times in the literature (see, for example, Deb, J. K et al. (1999) FEMS Microbiol. Lett. 175, 11-20).

It is likewise possible for genetic modification of corynebacteria to take place by modification of the DNA sequence of the genome. It is possible to introduce DNA sequences into the genome (newly introduced and/or introduction of further copies of sequences which are present), it is also possible to delete DNA sequence sections from the genome (e.g. genes or parts of genes), but it is also possible to carry out sequence exchanges (e.g. base exchanges) in the genome.

The modification of the genome can be achieved by introducing into the cell DNA which is preferably not replicated in the cell, and by recombining this introduced DNA with genomic host DNA and thus modifying the genomic DNA. This procedure is described, for example, in van der Rest, M. E. et al. (1999) Appl. Microbiol. Biotechnol. 52, 541-545 and references therein.

It is advantageous to be able to delete the transformation marker used (such as, for example, an antibiotic resistance gene) again because this marker can then be reused in further transformation experiments. One possibility for carrying this out is to use a marker gene which has a conditionally negatively dominant action.

A marker gene which has a conditionally negatively dominant action means a gene which is disadvantageous (e.g. toxic) for the host under certain conditions but has no adverse effects on the host harboring the gene under other conditions. An example from the literature is the URA3 gene from yeasts or fungi, an essential gene of pyrimidine biosynthesis which, however, is disadvantageous for the host if the chemical 5-fluoroorotic acid is present in the medium (see, for example, DE19801120, Rothstein, R. (1991) Methods in Enzymology 194, 281-301).

The use of a marker gene which has a conditionally negatively dominant action for deleting DNA sequences (for example the transformation marker used and/or vector sequences and other sequence sections), also called “pop-out”, is described, for example, in Schäfer et al. (1994) Gene 145, 69-73 or in Rothstein, R. (1991) Methods in Enzymology 194, 281-301.

Galactokinases (E.C.2.7.1.6) catalyze phosphorylation of galactose to give galactose phosphate. Numerous galactokinases from different organisms are known; thus, for example, the Escherichia coli galK gene (described by Debouck et al. (1985) Nucleic Acids Res. 13, 1841-1853), the Bacillus subtilis galK gene (Glaser et al. (1993) Mol. Microbiol. 10, 371-384) and the Saccharomyces cerevisiae GAL1 gene (Citron & Donelson (1984) J. Bacteriol. 158, 269-278) code in each case for a galactokinase.

Surprisingly, we have found that galactokinase genes are well suited to the use as marker genes which have a conditionally dominant negative action in Gram-positive bacteria, preferably corynebacteria. The galactokinase genes cause a sensitivity of corynebacteria to galactose in the nutrient medium (typically in a concentration range from 0.1 to 4% galactose in the medium).

The invention relates to a plasmid vector which does not replicate in a target organism, comprising the following components:

a) an origin of replication for a host organism which is different from the target organism,

b) at least one genetic marker,

c) where appropriate, a sequence section which makes possible the transfer of DNA via conjugation (mob sequence),

d) a sequence section which is homologous to sequences of the target organism and makes possible homologous recombination in the target organism,

e) a gene for a galactokinase under the control of a promotor.

Target organism means the organism which is to be genetically modified by the methods and plasmid vectors of the invention. Preferred organisms are Gram-positive bacteria, in particular bacteria strains from the genus Brevibacterium or Corynebacterium.

The promotor d) is preferably heterologous to the galactokinase gene used. Particularly suitable promotors are those from E. coli or C. glutamicum. Particular preference is given to the tac promotor.

The host organism in which the origin of replication a) is functionally active essentially serves for constructing and propagating the plasmid vector of the invention. Host organisms which may be used are all common microorganisms which can easily be manipulated by genetic engineering. Preferred host organisms are Gram-negative bacteria such as Escherichia coli or yeasts, for example Saccharomyces cerevisiae. The host organism must be genetically different from the target organism, since replication of the plasmid vector should not take place in the target organism but is desired in the host organism, due to using the origin of replication a).

Preference is given to exchanging in the target organism those sequences which are involved in an increase in the production of fine chemicals. Examples of those genes are given in WO 01/0842, 843 & 844, WO 01/0804 & 805, WO 01/2583.

Examples of alterations of this kind are genomic integrations of nucleic acid molecules (for example complete genes), disruptions (for example deletions or integrative disruptions) and sequence alterations (for example single or multiple point mutations, complete gene replacements). Preferred disruptions are those leading to a reduction in byproducts of the desired fermentation product, and preferred integrations are those enhancing a desired metabolism into a fermentation product and/or reducing or eliminating bottlenecks (de-bottlenecking). In the case of sequence alterations, appropriate metabolic adaptations are preferred. The fermentation product is preferably a fine chemical.

DNA may be transferred into the target organism by methods familiar to the skilled worker, preferably via conjugation or electroporation.

The DNA which is to be transferred into the target organism via conjugation contains specific sequence sections (called mob sequences hereinbelow) which makes this possible. Such mob sequences and their use for conjugation are described, for example, in Schäfer, A. et al. (1991) J. Bacteriol. 172, 1663-1666.

Genetic marker means a selectable property which is mediated by a gene. Preferred meanings are genes whose expression causes resistance to antibiotics, in particular a resistance to kanamycin, chloramphenicol, tetracycline or ampicillin.

Galactose-containing medium means in particular a medium containing at least 0.1% and not more than 10% (by weight) galactose.

Corynebacteria means for the purposes of the invention all Corynebacterium species, Brevibacterium species and Mycobacterium species. Preference is given to Corynebacterium species and Brevibacterium species.

Examples of Corynebacterium species and Brevibacterium species, which may be mentioned, are: Brevibacterium brevis, Brevibacterium lactofermentum, Corynebacterium ammoniagenes, Corynebacterium glutamicum, Corynebacterium diphtheriae, Corynebacterium lactofermentum.

Examples of Mycobacterium species are: Mycobacterium tuberculosis, Mycobacterium leprae, Mycobacterium bovis, Mycobacterium smegmatis.

Particularly preferred target organisms are those strains listed in the following table:

Table: Corynebacterium and Brevibacterium strains:

| Genus | species | ATCC | FERM | NRRL | CECT | NCIMB | CBS |
|---|---|---|---|---|---|---|---|
| Brevibacterium | ammoniagenes | 21054 | | | | | |
| Brevibacterium | ammoniagenes | 19350 | | | | | |
| Brevibacterium | ammoniagenes | 19351 | | | | | |
| Brevibacterium | ammoniagenes | 19352 | | | | | |
| Brevibacterium | ammoniagenes | 19353 | | | | | |
| Brevibacterium | ammoniagenes | 19354 | | | | | |
| Brevibacterium | ammoniagenes | 19355 | | | | | |
| Brevibacterium | ammoniagenes | 19356 | | | | | |
| Brevibacterium | ammoniagenes | 21055 | | | | | |
| Brevibacterium | ammoniagenes | 21077 | | | | | |
| Brevibacterium | ammoniagenes | 21553 | | | | | |
| Brevibacterium | ammoniagenes | 21580 | | | | | |
| Brevibacterium | ammoniagenes | 39101 | | | | | |
| Brevibacterium | butanicum | 21196 | | | | | |
| Brevibacterium | divaricatum | 21792 | P928 | | | | |
| Brevibacterium | flavum | 21474 | | | | | |
| Brevibacterium | flavum | 21129 | | | | | |
| Brevibacterium | flavum | 21518 | | | | | |
| Brevibacterium | flavum | | | B11474 | | | |
| Brevibacterium | flavum | | | B11472 | | | |
| Brevibacterium | flavum | 21127 | | | | | |
| Brevibacterium | flavum | 21128 | | | | | |
| Brevibacterium | flavum | 21427 | | | | | |
| Brevibacterium | flavum | 21475 | | | | | |
| Brevibacterium | flavum | 21517 | | | | | |
| Brevibacterium | flavum | 21528 | | | | | |
| Brevibacterium | flavum | 21529 | | | | | |
| Brevibacterium | flavum | | | B11477 | | | |
| Brevibacterium | flavum | | | B11478 | | | |
| Brevibacterium | flavum | 21127 | | | | | |
| Brevibacterium | flavum | | | B11474 | | | |
| Brevibacterium | healii | 15527 | | | | | |
| Brevibacterium | ketoglutamicum | 21004 | | | | | |
| Brevibacterium | ketoglutamicum | 21089 | | | | | |
| Brevibacterium | ketosoreductum | 21914 | | | | | |
| Brevibacterium | lactofermentum | | | | 70 | | |
| Brevibacterium | lactofermentum | | | | 74 | | |
| Brevibacterium | lactofermentum | | | | 77 | | |
| Brevibacterium | lactofermentum | 21798 | | | | | |
| Brevibacterium | lactofermentum | 21799 | | | | | |
| Brevibacterium | lactofermentum | 21800 | | | | | |
| Brevibacterium | lactofermentum | 21801 | | | | | |
| Brevibacterium | lactofermentum | | | B11470 | | | |
| Brevibacterium | lactofermentum | | | B11471 | | | |
| Brevibacterium | lactofermentum | 21086 | | | | | |
| Brevibacterium | lactofermentum | 21420 | | | | | |
| Brevibacterium | lactofermentum | 21086 | | | | | |
| Brevibacterium | lactofermentum | 31269 | | | | | |
| Brevibacterium | linens | 9174 | | | | | |
| Brevibacterium | linens | 19391 | | | | | |
| Brevibacterium | linens | 8377 | | | | | |
| Brevibacterium | paraffinolyticum | | | | | 11160 | |
| Brevibacterium | spec. | | | | | | 717.73 |
| Brevibacterium | spec. | | | | | | 717.73 |
| Brevibacterium | spec. | 14604 | | | | | |
| Brevibacterium | spec. | 21860 | | | | | |
| Brevibacterium | spec. | 21864 | | | | | |
| Brevibacterium | spec. | 21865 | | | | | |
| Brevibacterium | spec. | 21866 | | | | | |
| Brevibacterium | spec. | 19240 | | | | | |
| Corynebacterium | acetoacidophilum | 21476 | | | | | |
| Corynebacterium | acetoacidophilum | 13870 | | | | | |
| Corynebacterium | acetoglutamicum | | | B11473 | | | |
| Corynebacterium | acetoglutamicum | | | B11475 | | | |
| Corynebacterium | acetoglutamicum | 15806 | | | | | |
| Corynebacterium | acetoglutamicum | 21491 | | | | | |
| Corynebacterium | acetoglutamicum | 31270 | | | | | |
| Corynebacterium | acetophilum | | | B3671 | | | |
| Corynebacterium | ammoniagenes | 6872 | | | | | |
| Corynebacterium | ammoniagenes | 15511 | | | | | |
| Corynebacterium | fujiokense | 21496 | | | | | |
| Corynebacterium | glutamicum | 14067 | | | | | |
| Corynebacterium | glutamicum | 39137 | | | | | |
| Corynebacterium | glutamicum | 21254 | | | | | |
| Corynebacterium | glutamicum | 21255 | | | | | |
| Corynebacterium | glutamicum | 31830 | | | | | |
| Corynebacterium | glutamicum | 13032 | | | | | |
| Corynebacterium | glutamicum | 14305 | | | | | |
| Corynebacterium | glutamicum | 15455 | | | | | |
| Corynebacterium | glutamicum | 13058 | | | | | |
| Corynebacterium | glutamicum | 13059 | | | | | |
| Corynebacterium | glutamicum | 13060 | | | | | |
| Corynebacterium | glutamicum | 21492 | | | | | |
| Corynebacterium | glutamicum | 21513 | | | | | |
| Corynebacterium | glutamicum | 21526 | | | | | |
| Corynebacterium | glutamicum | 21543 | | | | | |
| Corynebacterium | glutamicum | 13287 | | | | | |
| Corynebacterium | glutamicum | 21851 | | | | | |
| Corynebacterium | glutamicum | 21253 | | | | | |
| Corynebacterium | glutamicum | 21514 | | | | | |
| Corynebacterium | glutamicum | 21516 | | | | | |
| Corynebacterium | glutamicum | 21299 | | | | | |
| Corynebacterium | glutamicum | 21300 | | | | | |
| Corynebacterium | glutamicum | 39684 | | | | | |
| Corynebacterium | glutamicum | 21488 | | | | | |
| Corynebacterium | glutamicum | 21649 | | | | | |
| Corynebacterium | glutamicum | 21650 | | | | | |
| Corynebacterium | glutamicum | 19223 | | | | | |
| Corynebacterium | glutamicum | 13869 | | | | | |
| Corynebacterium | glutamicum | 21157 | | | | | |
| Corynebacterium | glutamicum | 21158 | | | | | |
| Corynebacterium | glutamicum | 21159 | | | | | |
| Corynebacterium | glutamicum | 21355 | | | | | |
| Corynebacterium | glutamicum | 31808 | | | | | |
| Corynebacterium | glutamicum | 21674 | | | | | |
| Corynebacterium | glutamicum | 21562 | | | | | |
| Corynebacterium | glutamicum | 21563 | | | | | |
| Corynebacterium | glutamicum | 21564 | | | | | |
| Corynebacterium | glutamicum | 21565 | | | | | |
| Corynebacterium | glutamicum | 21566 | | | | | |
| Corynebacterium | glutamicum | 21567 | | | | | |
| Corynebacterium | glutamicum | 21568 | | | | | |
| Corynebacterium | glutamicum | 21569 | | | | | |
| Corynebacterium | glutamicum | 21570 | | | | | |
| Corynebacterium | glutamicum | 21571 | | | | | |
| Corynebacterium | glutamicum | 21572 | | | | | |
| Corynebacterium | glutamicum | 21573 | | | | | |
| Corynebacterium | glutamicum | 21579 | | | | | |
| Corynebacterium | glutamicum | 19049 | | | | | |
| Corynebacterium | glutamicum | 19050 | | | | | |
| Corynebacterium | glutamicum | 19051 | | | | | |
| Corynebacterium | glutamicum | 19052 | | | | | |
| Corynebacterium | glutamicum | 19053 | | | | | |
| Corynebacterium | glutamicum | 19054 | | | | | |
| Corynebacterium | glutamicum | 19055 | | | | | |
| Corynebacterium | glutamicum | 19056 | | | | | |
| Corynebacterium | glutamicum | 19057 | | | | | |
| Corynebacterium | glutamicum | 19058 | | | | | |
| Corynebacterium | glutamicum | 19059 | | | | | |
| Corynebacterium | glutamicum | 19060 | | | | | |
| Corynebacterium | glutamicum | 19185 | | | | | |
| Corynebacterium | glutamicum | 13286 | | | | | |
| Corynebacterium | glutamicum | 21515 | | | | | |
| Corynebacterium | glutamicum | 21527 | | | | | |
| Corynebacterium | glutamicum | 21544 | | | | | |
| Corynebacterium | glutamicum | 21492 | | | | | |
| Corynebacterium | glutamicum | | | B8183 | | | |
| Corynebacterium | glutamicum | | | B8182 | | | |
| Corynebacterium | glutamicum | | | B12416 | | | |
| Corynebacterium | glutamicum | | | B12417 | | | |
| Corynebacterium | glutamicum | | | B12418 | | | |
| Corynebacterium | glutamicum | | | B11476 | | | |
| Corynebacterium | glutamicum | 21608 | | | | | |
| Corynebacterium | lilium | | P973 | | | | |
| Corynebacterium | nitrilophilus | 21419 | | | | 11594 | |
| Corynebacterium | spec. | | P4445 | | | | |
| Corynebacterium | spec. | | P4446 | | | | |
| Corynebacterium | spec. | 31088 | | | | | |
| Corynebacterium | spec. | 31089 | | | | | |
| Corynebacterium | spec. | 31090 | | | | | |
| Corynebacterium | spec. | 31090 | | | | | |
| Corynebacterium | spec. | 31090 | | | | | |
| Corynebacterium | spec. | 15954 | | | | | |
| Corynebacterium | spec. | 21857 | | | | | |
| Corynebacterium | spec. | 21862 | | | | | |
| Corynebacterium | spec. | 21863 | | | | | |

ATCC: American Type Culture Collection, Rockville, MD, USA
FERM: Fermentation Research Institute, Chiba, Japan
NRRL: ARS Culture Collection, Northern Regional Research Laboratory, Peoria, IL, USA
CECT: Coleccion Espanola de Cultivos Tipo, Valencia, Spain
NCIMB: National Collection of Industrial and Marine Bacteria Ltd., Aberdeen, UK
CBS: Centraalbureau voor Schimmelcultures, Baarn, NL

The invention further relates to a method for preparing a marker-free mutated target organism, comprising the following steps:

a) transferring a plasmid vector as claimed in any of claims 1 to 10 into a target organism,

b) selecting clones of said target organism, which contain at least one genetic marker introduced by said plasmid vector,

c) selecting the clones of said target organism, obtained in step b), for the presence of galactose sensitivity by culturing in a galactose-containing medium.

The invention further relates to mutagenized Gram-positive bacteria (mutants), prepared using said method, in particular the mutagenized corynebacteria.

The mutants generated in this way may then be used for preparing fine chemicals or else, for example in the case of C. diphtheriae, for preparing, for example, vaccines with attenuated or nonpathogenic organisms.

Fine chemicals mean: organic acids, both proteinogenic and non-proteinogenic amino acids, nucleotides and nucleosides, lipids and fatty acids, diols, carbohydrates, aromatic compounds, vitamins and cofactors, and enzymes.

The term “fine chemical” is known in the art and comprises molecules which are produced by an organism and are used in various branches of industry such as, for example, but not restricted to, the pharmaceutical industry, the agricultural industry and the cosmetics industry. These compounds comprise organic acids such as tartaric acid, itaconic acid and diaminopimelic acid, both proteinogenic and nonproteinogenic amino acids, purine and pyrimidine bases, nucleosides and nucleotides (as described, for example, in Kuninaka, A. (1996) Nucleotides and related compounds, pp. 561-612, in Biotechnology Vol. 6, Rehm et al., editors VCH: Weinheim and the references therein), lipids, saturated and unsaturated fatty acids (for example arachidonic acid), diols (for example propanediol and butanediol), carbohydrates (for example hyaluronic acid and trehalose), aromatic compounds (for example aromatic amines, vanillin and indigo), vitamins and cofactors (as described in Ullmann's Encyclopedia of Industrial Chemistry, Vol. A27, “Vitamins”, pp. 443-613 (1996) VCH: Weinheim and the references therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held Sep. 1-3, 1994, in Penang, Malaysia, AOCS Press (1995)), Enzymes, Polyketides (Cane et al. (1998) Science 282: 63-68), and all other chemicals described by Gutcho (1983) in Chemicals by Fermentation, Noyes Data Corporation, ISBN: 0818805086 and the references indicated therein. The metabolism and the uses of certain fine chemicals are explained further below.

A. Amino Acid Metabolism and Uses

Amino acids comprise the fundamental structural units of all proteins and are thus essential for normal functions of the cell. The term “amino acid” is known in the art. Proteinogenic amino acids, of which there are 20 types, serve as structural units for proteins, in which they are linked together by peptide bonds, whereas the nonproteinogenic amino acids (hundreds of which are known) usually do not occur in proteins (see Ullmann's Encyclopedia of Industrial Chemistry, Vol. A2, pp. 57-97 VCH: Weinheim (1985)). Amino acids can exist in the D or L configuration, although L-amino acids are usually the only type found in naturally occurring proteins. Biosynthetic and degradation pathways of each of the 20 proteinogenic amino acids are well characterized both in prokaryotic and eukaryotic cells (see, for example, Stryer, L. Biochemistry, 3^{rd} edition, pp. 578-590 (1988)). The “essential” amino acids (histidine, isoleucine, leucine, lysine, methionine, phenylalanine, threonine, tryptophan and valine), so called because, owing to the complexity of their biosyntheses, they must be taken in with the diet, are converted by simple biosynthetic pathways into the other 11 “nonessential” amino acids (alanine, arginine, asparagine, aspartate, cysteine, glutamate, glutamine, glycine, proline, serine and tyrosine). Higher animals are able to synthesize some of these amino acids but the essential amino acids must be taken in with the food in order that normal protein synthesis takes place.

Apart from their function in protein biosynthesis, these amino acids are interesting chemicals as such, and it has been found that many have various applications in the human food, animal feed, chemicals, cosmetics, agricultural and pharmaceutical industries. Lysine is an important amino acid not only for human nutrition but also for monogastric livestock such as poultry and pigs. Glutamate is most frequently used as flavor additive (monosodium glutamate, MSG) and elsewhere in the food industry, as are aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D/L-methionine are widely used animal feed additives (Leuchtenberger, W. (1996) Amino acids—technical production and use, pp. 466-502 in Rehm et al., (editors) Biotechnology Vol. 6, Chapter 14a, VCH: Weinheim). It has been found that these amino acids are additionally suitable as precursors for synthesizing synthetic amino acids and proteins, such as N-acetylcysteine, S-carboxymethyl-L-cysteine, (S)-5-hydroxytryptophan and other substances described in Ullmann's Encyclopedia of Industrial Chemistry, Vol. A2, pp. 57-97, VCH, Weinheim, 1985.

The biosynthesis of these natural amino acids in organisms able to produce them, for example bacteria, has been well characterized (for a review of bacterial amino acid biosynthesis and its regulation, see Umbarger, H. E. (1978) Ann. Rev. Biochem. 47: 533-606). Glutamate is synthesized by reductive amination of α-ketoglutarate, an intermediate product in the citric acid cycle. Glutamine, proline and arginine are each generated successively from glutamate. The biosynthesis of serine takes place in a three-step process and starts with 3-phosphoglycerate (an intermediate product of glycolysis), and affords this amino acid after oxidation, transamination and hydrolysis steps. Cysteine and glycine are each produced from serine, specifically the former by condensation of homocysteine with serine, and the latter by transfer of the side-chain β-carbon atom to tetrahydrofolate in a reaction catalyzed by serine transhydroxymethylase. Phenylalanine and tyrosine are synthesized from the precursors of the glycolysis and pentose phosphate pathway, and erythrose 4-phosphate and phosphoenolpyruvate in a 9-step biosynthetic pathway which diverges only in the last two steps after the synthesis of prephenate. Tryptophan is likewise produced from these two starting molecules but it is synthesized by an 11-step pathway. Tyrosine can also be prepared from phenylalanine in a reaction catalyzed by phenylalanine hydroxylase. Alanine, valine and leucine are each biosynthetic products derived from pyruvate, the final product of glycolysis. Aspartate is formed from oxalacetate, an intermediate product of the citrate cycle. Asparagine, methionine, threonine and lysine are each produced by the conversion of aspartate. Isoleucine is formed from threonine. Histidine is formed from 5-phosphoribosyl 1-pyrophosphate, an activated sugar, in a complex 9-step pathway.

Amounts of amino acids exceeding those required for protein biosynthesis by the cell cannot be stored and are instead broken down so that intermediate products are provided for the principal metabolic pathways in the cell (for a review, see Stryer, L., Biochemistry, 3^{rd} edition, Chapter 21 “Amino Acid Degradation and the Urea Cycle”; pp. 495-516 (1988)). Although the cell is able to convert unwanted amino acids into the useful intermediate products of metabolism, production of amino acids is costly in terms of energy, the precursor molecules and the enzymes necessary for their synthesis. It is therefore not surprising that amino acid biosynthesis is regulated by feedback inhibition, whereby the presence of a particular amino acid slows down or completely stops its own production (for a review of the feedback mechanism in amino acid biosynthetic pathways, see Stryer, L., Biochemistry, 3^{rd} edition, Chapter 24, “Biosynthesis of Amino Acids and Heme”, pp. 575-600 (1988)). The output of a particular amino acid is therefore restricted by the amount of this amino acid in the cell.

B. Vitamins, Cofactors and Nutraceutical Metabolism, and Uses

Vitamins, cofactors and nutraceuticals comprise another group of molecules. Higher animals have lost the ability to synthesize them and therefore have to take them in, although they are easily synthesized by other organisms such as bacteria. These molecules are either bioactive molecules per se or precursors of bioactive substances which serve as electron carriers or intermediate products in a number of metabolic pathways. Besides their nutritional value, these compounds also have a significant industrial value as colorants, antioxidants and catalysts or other processing auxiliaries. (For a review of the structure, activity and industrial applications of these compounds, see, for example, Ullmann's Encyclopedia of Industrial Chemistry, “Vitamins”, Vol. A27, pp. 443-613, VCH: Weinheim, 1996). The term “vitamin” is known in the art and comprises nutrients which are required for normal functional of an organism but cannot be synthesized by this organism itself. The group of vitamins may include cofactors and nutraceutical compounds. The term “cofactor” comprises nonproteinaceous compounds necessary for the appearance of a normal enzymic activity. These compounds may be organic or inorganic; the cofactor molecules of the invention are preferably organic. The term “nutraceutical” comprises food additives which are health-promoting in plants and animals, especially humans. Examples of such molecules are vitamins, antioxidants and likewise certain lipids (e.g. polyunsaturated fatty acids).

The biosynthesis of these molecules in organisms able to produce them, such as bacteria, has been comprehensively characterized (Ullmann's Encyclopedia of Industrial Chemistry, “Vitamins”, Vol. A27, pp. 443-613, VCH: Weinheim, 1996, Michal, G. (1999) Biochemical Pathways: An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research—Asia, held on Sep. 1-3, 1994, in Penang, Malaysia, AOCS Press, Champaign, IL X, 374 S).

Thiamine (vitamin B₁) is formed by chemical coupling of pyrimidine and thiazole units. Riboflavin (vitamin B₂) is synthesized from guanosine 5′-triphosphate (GTP) and ribose 5′-phosphate. Riboflavin in turn is employed for the synthesis of flavin mononucleotide (FMN) and flavin adenine dinucleotide (FAD). The family of compounds together referred to as “vitamin B6” (for example pyridoxine, pyridoxamine, pyridoxal 5′-phosphate and the commercially used pyridoxine hydrochloride), are all derivatives of the common structural unit 5-hydroxy-6-methylpyridine. Pantothenate (pantothenic acid, R-(+)-N-(2,4-dihydroxy-3,3-dimethyl-1-oxobutyl)-β-alanine) can be prepared either by chemical synthesis or by fermentation. The last steps in pantothenate biosynthesis consist of ATP-driven condensation of β-alanine and pantoic acid. The enzymes responsible for the biosynthetic steps for the conversion into pantoic acid and into β-alanine and for the condensation to pantothenic acid are known. The metabolically active form of pantothenate is coenzyme A whose biosynthesis takes place by 5 enzymatic steps. Pantothenate, pyridoxal 5′-phosphate, cysteine and ATP are the precursors of coenzyme A. These enzymes catalyze not only the formation of pantothenate but also the production of (R)-pantoic acid, (R)-pantolactone, (R)-panthenol (provitamin B₅), pantetheine (and its derivatives) and coenzyme A.

The biosynthesis of biotin from the precursor molecule pimeloyl-CoA in microorganisms has been investigated in detail, and several of the genes involved have been identified. It has emerged that many of the corresponding proteins are involved in the Fe cluster synthesis and belong to the class of nifS proteins. Liponic acid is derived from octanoic acid and serves as coenzyme in energy metabolism where it is a constituent of the pyruvate dehydrogenase complex and of the α-ketoglutarate dehydrogenase complex. Folates are a group of substances all derived from folic acid which in turn is derived from L-glutamic acid, p-aminobenzoic acid and 6-methylpterin. The biosynthesis of folic acid and its derivatives starting from the metabolic intermediate products of guanosine 5′-triphosphate (GTP), L-glutamic acid and p-aminobenzoic acid has been investigated in detail in certain microorganisms.

Corrinoids (such as the cobalamines and, in particular, vitamin B₁₂) and the porphyrins belong to a group of chemicals distinguished by a tetrapyrrole ring system. The biosynthesis of vitamin B₁₂ is so complex that it has not yet been completely characterized, but most of the enzymes and substrates involved are now known. Nicotinic acid (nicotinate) and nicotinamide are pyridine derivatives which are also referred to as “niacin”. Niacin is the precursor of the important coenzymes NAD (nicotinamide adenine dinucleotide) and NADP (nicotinamide adenine dinucleotide phosphate) and their reduced forms.

Production of these compounds on the industrial scale is mostly based on cell-free chemical syntheses, although some of these chemicals have likewise been produced by large-scale cultivation of microorganisms, such as riboflavin, vitamin B₆, pantothenate and biotin. Only vitamin B₁₂ is, because of the complexity of its synthesis, produced only by fermentation. In vitro processes require a considerable expenditure of materials and time and frequently high costs.

C. Purine, Pyrimidine, Nucleoside and Nucleotide Metabolism and Uses

Genes for purine and pyrimidine metabolism and their corresponding proteins are important aims for the therapy of oncoses and viral infections. The term “purine” or “pyrimidine” comprises nitrogen-containing bases which form part of nucleic acids, coenzymes and nucleotides. The term “nucleotide” encompasses the fundamental structural units of nucleic acid molecules, which comprise a nitrogen-containing base, a pentose sugar (the sugar is ribose in the case of RNA and the sugar is D-deoxyribose in the case of DNA) and phosphoric acid. The term “nucleoside” comprises molecules which serve as precursors of nucleotides but have, in contrast to the nucleotides, no phosphoric acid unit. It is possible to inhibit RNA and DNA synthesis by inhibiting the biosynthesis of these molecules or their mobilization to form nucleic acid molecules; targeted inhibition of this activity in cancerous cells allows the ability of tumor cells to divide and replicate to be inhibited.

There are also nucleotides which do not form nucleic acid molecules but serve as energy stores (i.e. AMP) or as coenzymes (i.e. FAD and NAD).

Several publications have described the use of these chemicals for these medical indications, the purine and/or pyrimidine metabolism being influenced (for example Christopherson, R. I. and Lyons, S. D. (1990) “Potent inhibitors of de novo pyrimidine and purine biosynthesis as chemotherapeutic agents”, Med. Res. Reviews 10: 505-548). Investigations of enzymes involved in purine and pyrimidine metabolism have concentrated on the development of novel medicaments which can be used, for example, as immunosuppressants or antiproliferative agents (Smith, J. L. “Enzymes in Nucleotide Synthesis” Curr. Opin. Struct. Biol. 5 (1995) 752-757; Simmonds, H. A., Biochem. Soc. Transact. 23 (1995) 877-902). However, purine and pyrimidine bases, nucleosides and nucleotides also have other possible uses: as intermediate products in the biosynthesis of various fine chemicals (e.g. thiamine, S-adenosylmethionine, folates or riboflavin), as energy carriers for the cell (for example ATP or GTP) and for chemicals themselves, are ordinarily used as flavor enhancers (for example IMP or GMP) or for many medical applications (see, for example, Kuninaka, A., (1996) “Nucleotides and Related Compounds in Biotechnology” Vol. 6, Rehm et al., editors VCH: Weinheim, pp. 561-612). Enzymes involved in purine, pyrimidine, nucleoside or nucleotide metabolism are also increasingly serving as targets against which chemicals are being developed for crop protection, including fungicides, herbicides and insecticides.

The metabolism of these compounds in bacteria has been characterized (for reviews, see, for example, Zalkin, H. and Dixon, J. E. (1992) “De novo purine nucleotide biosynthesis” in Progress in Nucleic Acids Research and Molecular biology, Vol. 42, Academic Press, pp. 259-287; and Michal, G. (1999) “Nucleotides and Nucleosides”; Chapter 8 in: Biochemical Pathways: An Atlas of Biochemistry and Molecular Biology, Wiley, New York). Purine metabolism, the object of intensive research, is essential for normal functioning of the cell. Disordered purine metabolism in higher animals may cause severe illnesses, for example gout. Purine nucleotides are synthesized from ribose 5-phosphate by a number of steps via the intermediate compound inosine 5′-phosphate (IMP), leading to the production of guanosine 5′-monophosphate (GMP) or adenosine 5′-monophosphate (AMP), from which the triphosphate forms used as nucleotides can easily be prepared. These compounds are also used as energy stores, so that breakdown thereof provides energy for many different biochemical processes in the cell. Pyrimidine biosynthesis takes place via formation of uridine 5′-monophosphate (UMP) from ribose 5-phosphate. UMP in turn is converted into cytidine 5′-triphosphate (CTP). The deoxy forms of all nucleotides are prepared in a one-step reduction reaction from the diphosphate ribose form of the nucleotide to give the diphosphate deoxyribose form of the nucleotide. After phosphorylation, these molecules can take part in DNA synthesis.

D. Trehalose Metabolism and Uses

Trehalose consists of two glucose molecules linked together by α,α-1,1 linkage. It is ordinarily used in the food industry as sweetener, as additive for dried or frozen foods and in beverages. However, it is also used in the pharmaceutical industry or in the cosmetics industry and biotechnology industry (see, for example, Nishimoto et al., (1998) U.S. Pat. No. 5,759,610; Singer, M. A. and Lindquist, S. Trends Biotech. 16 (1998) 460-467; Paiva, C. L. A. and Panek, A. D. Biotech Ann. Rev. 2 (1996) 293-314; and Shiosaka, M. J. Japan 172 (1997) 97-102). Trehalose is produced by enzymes of many microorganisms and is naturally released into the surrounding medium from which it can be isolated by methods known in the art.

### EXAMPLE 1

PCR Cloning of the Galactokinase Gene galK9 from Escherichia coli C600.

Primers which may be used for cloning the E. coli galactokinase gene via PCR are oligonucleotides which can be defined on the basis of the published galactokinase sequences (for example GenBank entry X02306). The PCR template (E. coli genomic DNA) may be prepared and the PCR may be carried out according to methods which are well-known to the skilled worker and are described, for example, in Sambrook, J. et al. (1989) “Molecular Cloning: A Laboratory Manual”, Cold Spring Harbor Laboratory Press or Ausubel, F. M. et al. (1994) “Current Protocols in Molecular Biology”, John Wiley & Sons. The galactokinase gene (galK gene), consisting of the protein-encoding sequence and 30 bp of sequences located 5′ of the coding sequence (ribosomal binding site), can be provided with terminal cleavage sites for restriction end nucleases (for example EcoRI) during the course of the PCR, and the PCR product can then be cloned into suitable vectors (such as plasmids pUC18 or pWST4B (Liebl et al. (1989) FEMS Microbiol. Lett. 65, 299-304)) which comprise suitable cleavage sites for restriction end nucleases. This method of cloning genes via PCR is known to the skilled worker and is described, for example, in Sambrook, J. et al. (1989) “Molecular Cloning: A Laboratory Manual”, Cold Spring Harbor Laboratory Press or Ausubel, F. M. et al. (1994) “Current Protocols in Molecular Biology”, John Wiley & Sons. Cloning of the E. coli galK gene with the known sequence can be detected by sequence analysis.

### EXAMPLE 2

Assay of galK-mediated Galactose Sensitivity in Corynebacterium glutamicum R163

Corynebacterium glutamicum R163 is described, for example, in Liebl et al. (1992) J. Bacteriol. 174, 1854-1861. The E. coli galK gene was first put under the control of a heterologous promotor. For this purpose, the E. coli tac promotor was cloned using PCR methods.

The tac promotor and the galK gene were then cloned into plasmid pWST4B (Liebl et al. (1989) FEMS Microbiol. Lett. 65, 299-304), a shuttle vector which can replicate both in E. coli and in C. glutamicum and mediates chloramphenicol resistance. After DNA transfer into C. glutamicum (see, for example, WO 01/02583) and selection of chloramphenicol-resistant colonies, said colonies were tested for galactose sensitivity. For this purpose, cells were streaked out on LB medium (10 g/l peptone, 5 g/l yeast extract, 5 g/l NaCl, 12 g/l Agar, pH 7.2) which have been supplemented with Chloramphenicol (5 mg/l) or with Chloramphenicol (5 mg/l) and galactose (0.8%). Clones expressing the galK gene were grown overnight only on galactose-free plates.

### EXAMPLE 3

Inactivation of the ddh Gene from Corynebacterium glutamicum

Any suitable sequence section at the 5′ end of the ddh gene of C. glutamicum (Ishino et al.(1987) Nucleic Acids Res. 15, 3917) and any suitable sequence section at the 3′ end of the ddh gene can be amplified by known PCR methods. The two PCR products can be fused by known methods so that the resulting product has no functional ddh gene. This inactive form of the ddh gene, and the galk gene from E. coli, can be cloned into pSL18 (Kim, Y. H. & H.-S. Lee (1996) J. Microbiol. Biotechnol. 6, 315-320) to result in the vector pSL18galkΔddh. The procedure is familiar to the skilled worker. Transfer of this vector into Corynebacterium is known to the skilled worker and is possible, for example, by conjugation or electroporation.

Selection of the integrants can take place with kanamycin, and selection for the “pop-out” can take place as described in Example 2. Inactivation of the ddh gene can be shown, for example, by the lack of Ddh activity. Ddh activity can be measured by known methods (see, for example, Misono et al. (1986) Agric. Biol. Chem. 50, 1329-1330).

We claim:

1. A plasmid vector which does not replicate in a target organism, comprising the following components:
a) an origin of replication for a host organism which is different from the target organism,
b) at least one genetic marker,
c) where appropriate, a sequence section which makes possible the transfer of DNA via conjugation (mob sequence),
d) a sequence section which is homologous to sequences of the target organism and makes possible homologous recombination in the target organism,
e) a gene for a galactokinase under the control of a promotor.

2. A plasmid vector as claimed in claim 1, whose host organism a) is Escherichia coli.

3. A plasmid vector as claimed in claim 1, wherein the galactokinase gene is from Escherichia coli.

4. A plasmid vector as claimed in claim 1, wherein the genetic marker b) imparts a resistance to antibiotics.

5. A plasmid vector as claimed in claim 1, wherein the promotor e) is heterologous.

6. A plasmid vector as claimed in claim 1, which contains the sequence section c).

7. A plasmid vector as claimed in claim 4, which imparts a resistance to kanamycin, chloramphenicol, tetracycline or ampicillin.

8. A plasmid vector as claimed in claim 5, wherein the heterologous promotor is from E. coli or C. glutamicum.

9. A plasmid vector as claimed in claim 5, wherein the heterologous promotor is a tac promotor.

10. A method for preparing a marker-free mutated target organism, comprising the following steps:
a) transferring a plasmid vector as claimed in any of claims 1 to 10 into a target organism,
b) selecting clones of said target organism, which contain at least one genetic marker introduced by said plasmid vector,
c) selecting the clones of said target organism, obtained in step b), for the presence of galactose sensitivity by culturing in a galactose-containing medium.

11. A method as claimed in claim 10, wherein the target organism is a Gram-positive bacterial strain.

12. A method as claimed in claim 11, wherein the target organism is a bacterial strain of the genus Brevibacterium or Corynebacterium.

13. A method as claimed in claim 10, wherein the DNA is transferred via conjugation or electroporation.

14. A mutagenized Gram-positive bacterium, obtainable according to a method as claimed in claim 11.

15. The use of a galactokinase gene as conditionally negatively dominant marker gene.
